(19) United States
(12) Patent Application Publication
(10) Pub. No.: US 2025/0205092 A1
(43) Pub. Date: Jun. 26, 2025

(54) DISPOSABLE ABSORBENT PRODUCT

(71) Applicant: LIVEDO CORPORATION, Ehime (JP)

(72) Inventors: Keiichi ISHIKAWA, Tokushima (JP); Osamu MORIURA, Tokushima (JP)

(73) Assignee: LIVEDO CORPORATION, Ehime (JP)

(21) Appl. No.: 18/848,695

(22) PCT Filed: Jul. 27, 2022
(86) PCT No.: PCT/JP2022/028975

§ 371 Date: Sep. 19, 2024

(30) Foreign Application Priority Data
Mar. 25, 2022 (JP) 2022-049269

(51) Int. Cl. A61F 13/49; A61F 13/15

(52) U.S. Cl. CPC A61F 13/49015; A61F 13/15739; A61F 2013/49093

(57) ABSTRACT

In a disposable absorbent product (1), a center sheet (31) and a pair of gather sheets (32) are joined together in a compression joining region (4) by welding involving pressure. The compression joining region (4) includes a central joining region (41) positioned in the range of a central portion in a longitudinal direction, and end joining regions (42) positioned in the ranges of both end portions in the longitudinal direction. The end joining regions (42) each include an outer joining region (432) that exists outward of elastic members (3222, 3223) of one gather sheet (32) in a width direction and an inner joining region (431) that exists inward of the elastic members (3222, 3223) in the width direction, and do not overlap with the elastic members (3221 to 3223).

### CROSS-REFERENCE TO RELATED APPLICATIONS

This application claims priority benefit of Japanese Patent Application No. JP2022-049269 filed in the Japan Patent Office on Mar. 25, 2022, the entire disclosure of which is incorporated herein by reference.

### TECHNICAL FIELD

The present invention relates to a disposable absorbent product that receives body wastes from a wearer.

### BACKGROUND ART

Conventionally, techniques are known in which nonwoven fabrics are joined together by welding that applies pressure while using heat or ultrasonic vibrations. Joints produced by such techniques are also called “embossments.” Japanese Patent Application Laid-Open No. 2008-289622 (Document 1) discloses a feminine hygiene napkin in which side sheets are arranged to cover both side portions of a surface sheet, and the surface sheet and the side sheets are joined together by embossed portions that extend in a back-and-forth direction. The embossed portions have a wide width in the front and rear parts of the feminine hygiene napkin and have a narrow width in the central portion.

Japanese Patent Application Laid-Open No. 2003-24384 (Document 2) discloses a structure in which joints between a surface sheet and anti-leak portions in the central part of a feminine hygiene napkin are positioned more outward in the width direction than those in the front and rear parts.

Meanwhile, regions where embossments are formed become relatively hardened. Thus, in the case where regions in which embossments are formed exist largely apart from side to side in the front and rear parts of an absorbent product as in Document 1, a user feels uncomfortable with right and left side portions in the front and rear parts of the absorbent product. In particular, in the case where gather sheets for preventing leakage exist widely on the berry side and the back side of an absorbent product when the absorbent product is worn as in the case of a disposable diaper or a disposable auxiliary absorbent pad arranged on the inside such as a diaper or a diaper-shaped cover, a user is likely to feel discomfort if the gather sheets are to be fixedly attached to the center sheet by embossing (i.e., welding). In Document 1, no consideration is given to such discomfort caused by the right and left side portions in the front and rear parts of the absorbent product when the gather sheets including elastic members are provided. In the feminine hygiene napkin according to Document 1, the embossed portions exist linearly in almost the longitudinal direction, and such a structure that causes gathers to stand up significantly is not employed.

### SUMMARY OF THE INVENTION

It is an object of the present invention to improve the wearing comfort of an absorbent product in which gather sheets are joined to a center sheet by welding involving pressure.

A first aspect of the present invention is a disposable absorbent product for receiving a body wastes from a wearer. The disposable absorbent product includes a top sheet, a back sheet, and an absorbent core positioned between the top sheet and the back sheet. The top sheet includes a center sheet and a pair of gather sheets fixedly attached to the center sheet while being spaced apart from a center line extending in a longitudinal direction on both sides in a width direction perpendicular to the longitudinal direction. Each of the pair of gather sheets includes a side sheet that is long in the longitudinal direction and an elastic member extending in the longitudinal direction and fixedly attached to a region on an inner side in the width direction of the side sheet. The center sheet and the pair of gather sheets are joined together in a compression joining region by welding involving pressure. The compression joining region between the center sheet and each gather sheet includes a central joining region positioned in a central range in the longitudinal direction and spaced outward in the width direction from the elastic member, and an end joining region that is positioned in ranges of both end portions in the longitudinal direction and does not overlap with the elastic member. The end joining region includes an outer joining region located outward of the elastic member in the width direction and an inner joining region located inward of the elastic member in the width direction.

According to the present invention, it is possible to improve the wearing comfort of the absorbent product in which the gather sheets are joined to the center sheet by welding involving pressure.

A second aspect of the present invention is the disposable absorbent product according to the first aspect, in which the central joining region and the outer joining region are continuous in the longitudinal direction.

A third aspect of the present invention is the disposable absorbent product according to the first or second aspect, in which an outer edge of the end joining region in the width direction is located inward in the width direction of an inner edge in the width direction of the central joining region.

A fourth aspect of the present invention is the disposable absorbent product according to the first or second aspect (or any one of the first to third aspects), the compression joining region is an assembly of a large number of dot-shaped joining elements, and a ratio of existence of joining elements between outer and inner edges in the width direction of the end joining region is lower than a ratio of existence of joining elements between outer and inner edges in the width direction of the central joining region.

A fifth aspect of the present invention is the disposable absorbent product according to the first or second aspect (or any one of the first to fourth aspects) that includes a plurality of elastic members including the elastic member. An elastic member positioned on an innermost side in the width direction among the plurality of elastic members is positioned inward in the width direction of the inner joining region.

A sixth aspect of the present invention is the disposable absorbent product according to the fifth aspect, in which an elastic member positioned on a second innermost side in the width direction among the plurality of elastic members is positioned between the inner joining region and the outer joining region in the width direction.

A seventh aspect of the present invention is the disposable absorbent product according to the first or second aspect (or any one of the first to sixth aspects), in which the outer joining region includes a plurality of linear regions extending in the longitudinal direction.

An eighth aspect of the present invention is the disposable absorbent product according to the first or second aspect (or any one of the first to seventh aspects), in which the compression joining region exists along an overall length in the longitudinal direction of the top sheet, and a maximum value of a total length of a compression joint in the width direction of the compression joining region is 1.25 time or more and 4 times or less of a minimum value of the total length.

A ninth aspect of the present invention is the disposable absorbent product according to the first or second aspect (or any one of the first to eighth aspects), in which an edge of the end joining region on a central side in the longitudinal direction is a discontinuous line, the edge is inclined toward a center in the longitudinal direction toward an outside in the width direction, and an additional joining region exists on an outer or inner side in the width direction where the edge is interrupted.

A tenth aspect of the present invention is the disposable absorbent product according to the first or second aspect (or any one of the first to ninth aspects) that is arranged in a diaper or in a diaper-shaped cover.

These and other objects, features, aspects and advantages of the present invention will become more apparent from the following detailed description of the present invention when taken in conjunction with the accompanying drawings.

### BRIEF DESCRIPTION OF DRAWINGS

FIG. 1 is a plan view showing a configuration of an absorbent product.

FIG. 2 is a perspective view showing one free state of the absorbent product.

FIG. 3 is a sectional view of the absorbent product.

FIG. 4 is a sectional view of the absorbent product.

FIG. 5 is a sectional view showing the vicinity of a gather sheet in enlarged dimensions.

FIG. 6 is a diagram showing the vicinity of a boundary between a central joining region and an end joining region in enlarged dimensions.

### DESCRIPTION OF EMBODIMENTS

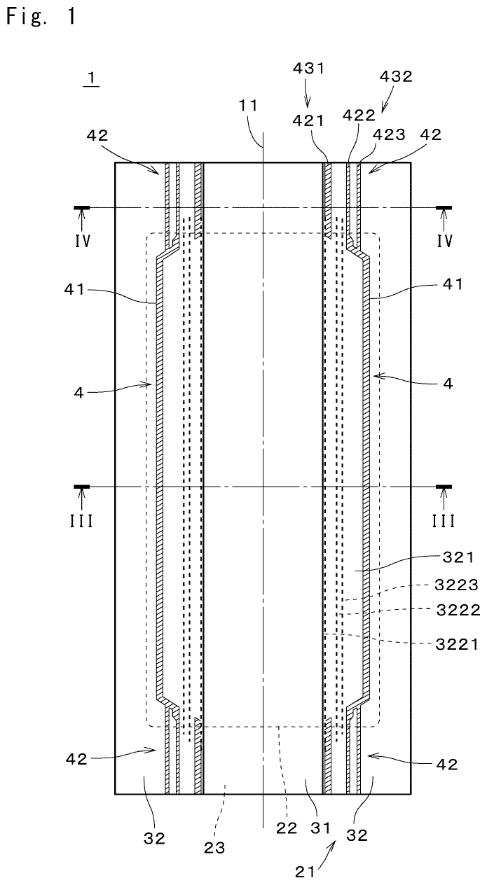

FIG. 1 is a plan view showing a configuration of a disposable absorbent product 1 (hereinafter, also simply referred to as the “absorbent product 1”) according to one embodiment of the present invention. FIG. 1 shows a state in which the absorbent product 1 is opened in a planar state. The absorbent product 1 is an auxiliary absorbent pad that is arranged on the inner side (i.e., wearer side) of an exterior product worn by a wearer to receive body wastes from the wearer. Examples of the exterior product include a disposable diaper, a non-disposable diaper, and a diaper-shaped cover. For example, the absorbent product 1 may be used for a person who gets care in a hospital or a nursing-care facility. In FIG. 1, the surface of the absorbent product 1 on the side that comes in contact with the wearer is shown toward the front. In the following description, the up-down direction in FIG. 1 is referred to as the “longitudinal direction,” and the right-left direction perpendicular to the longitudinal direction is referred to as the “width direction.” The absorbent product 1 is bilaterally symmetrical, and the center line that extends in the longitudinal direction in FIG. 1 is given a reference sign 11.

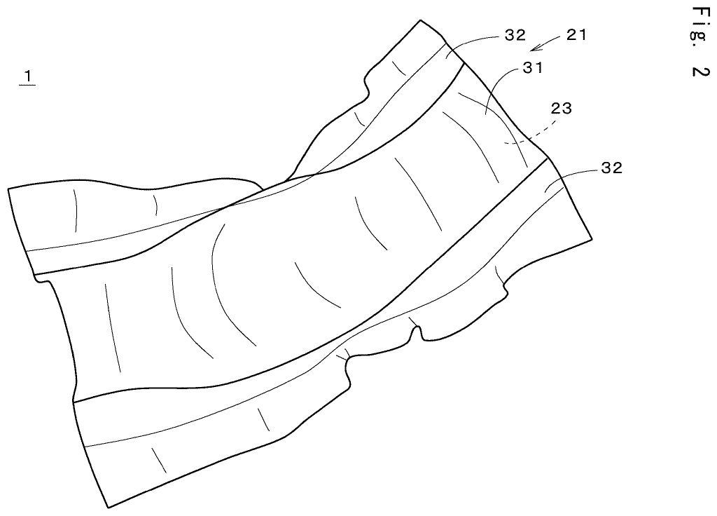

FIG. 2 is a perspective view showing one free state of the absorbent product 1. As will be described later, the absorbent product 1 includes elastic members and are curved as a whole due to shrinkage of the elastic members.

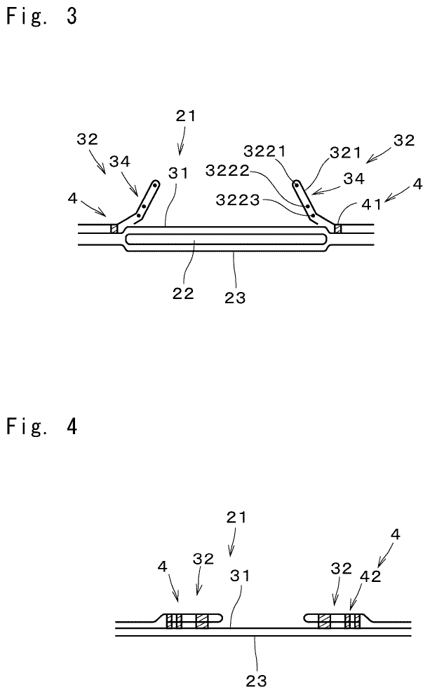

FIG. 3 is a sectional view of the absorbent product 1, taken along a plane perpendicular to the longitudinal direction at a position III-III in FIG. 1. Since gather sheets described later have gathers that stand up due to a shrinkage force of the elastic members as shown in FIG. 2, FIG. 3 shows a state in which the gathers are standing. FIG. 4 is a sectional view of the absorbent product 1, taken along a plane perpendicular to the longitudinal direction at a position IV-IV in FIG. 1.

As shown in FIGS. 1 to 4, the absorbent product 1 includes a top sheet 21, an absorbent core 22, and a back sheet 23. The absorbent core 22 is positioned between the top sheet 21 and the back sheet 23. The top sheet 21 includes a center sheet 31 and a pair of gather sheets 32. The pair of gather sheets 32 is fixedly attached on both side portions of the center sheet 31 (i.e., at spaced positions on both sides in the width direction) and extends in the longitudinal direction. Each gather sheet 32 exists along the overall longitudinal length of the center sheet 31. In other words, the longitudinal length of each gather sheet 32 is substantially the same as the longitudinal length of the absorbent product 1. Inner portions of the pair of gather sheets 32 in the width direction, i.e., gathers 34, stand up toward the wearer when the absorbent product 1 is worn.

In FIG. 1, an upper portion and a lower portion of the absorbent product 1 correspond respectively to a front part that comes in contact with the skin on the belly side of the wearer and a rear part that comes in contact with the skin on the back side of the wearer. A portion of the absorbent product 1 that is positioned between and continuous from the front part and the rear part is a central part that faces a groin of the wearer. There is no need to accurately determine boundaries between the front part and the central part and between the rear part and the central part. The front, central, and rear parts of the absorbent product 1 are arranged in this order in the longitudinal direction. In the example shown in FIG. 1, the absorbent product 1 has an approximately rectangular shape in plan view, and the front, central, and rear parts have substantially the same width in the width direction (i.e., the width in the right-left direction when the absorbent product 1 is worn; hereinafter also simply referred to as the “width”). In other words, the absorbent product 1 has an approximately constant width along its overall longitudinal length. FIGS. 3 and 4 described above are sectional views of the central part and the front part of the absorbent product 1, respectively. A section of the rear part of the absorbent product 1 is substantially similar to the section of the front part shown in FIG. 4. The width of the absorbent product 1 does not necessarily have to be constant, and for example, the width of the rear part may be greater than the width of the front part.

As shown in FIGS. 1, 3, and 4, the absorbent core 22 is sandwiched between the top sheet 21 and the back sheet 23. The top sheet 21 and the back sheet 23 are joined together around the absorbent core 22 with an adhesive. In FIG. 1, the contour of the absorbent core 22 is illustrated with a thin broken line. In FIGS. 3 and 4, to facilitate understanding of the drawings, the absorbent product 1 is illustrated greater than the actual dimension in the thickness direction (i.e., the up-down direction in FIGS. 3 and 4), and constituent elements of the absorbent product 1 that are in contact with each other or are joined together in reality are illustrated with appropriate spaces in the thickness direction.

The center sheet 31 of the top sheet 21 speedily captures moisture from body wastes received from the wearer and transmits the moisture to the absorbent core 22. The absorbent core 22 absorbs the moisture transmitted through the center sheet 31 and speedily immobilizes the moisture. The back sheet 23 prevents the moisture or the like in the body wastes most of which are transmitted through the center sheet 31 and the absorbent core 22 to the back sheet 23 from leaking to the outside of the absorbent product 1. In the example shown in FIG. 3, the absorbent core 22 is in contact with the center sheet 31 and the back sheet 23, but there may be a mount (e.g., tissue paper) arranged between the absorbent core 22 and the center sheet 31 or between the absorbent core 22 and the back sheet 23. The absorbent core 22 may be enclosed by any other sheet, or may be obtained by overlaying a plurality of absorbent cores one above another. That is, the absorbent core 22 and its peripheral structure may adopt any of various known structures.

The longitudinal lengths of the top sheet 21 and the back sheet 23 are greater than the length of the absorbent core 22. The widths of the top sheet 21 and the back sheet 23 in the width direction are greater than the width of the absorbent core 22. Both side portions of the top sheet 21 (i.e., portions on both sides in the width direction) are joined with both side portions of the back sheet 23 either directly or indirectly via an inclusion along the overall length. The width of the center sheet 31 may be smaller than the width of the back sheet 23. In this case, outer end portions of the gather sheet 32 in the width direction may be joined with the back sheet 23. In the longitudinal end portions of the absorbent product 1, the longitudinal end portions of the top sheet 21 are joined either directly or indirectly with the longitudinal end portions of the back sheet 23. The top sheet 21 and the back sheet 23 are joined together along the entire periphery of the absorbent core 22 and accordingly the absorbent core 22 as a whole is surrounded by the top sheet 21 and the back sheet 23. For example, the top sheet 21 and the back sheet 23 may be joined together with a hot-melt adhesive. The top sheet 21 and the back sheet 23 may be joined together by any of various methods other than using the hot-melt adhesive.

As the center sheet 31, for example, a liquid-permeable nonwoven fabric (e.g., a point-bonded nonwoven fabric, an air-through nonwoven fabric, or a spun-bonded nonwoven fabric) may be used that is formed of hydrophobic fiber (e.g., polypropylene, polyethylene, polyester, polyamide, or nylon) having a surface subjected to hydrophilic treatment using a surface-active agent. Note that the center sheet 31 may also be a nonwoven fabric (e.g., a spun lace nonwoven fabric) formed of hydrophilic fiber such as cellulose, rayon, or cotton.

Preferably, the back sheet 23 may have a layered structure in which a plurality of sheets are overlaid one above another. For example, the back sheet 23 may have a structure in which a nonwoven fabric is overlaid on a plastic film or a water-repellent or liquid-impermeable nonwoven fabric. In the case where the back sheet 23 includes a plastic film, the plastic film is arranged so as to face the absorbent core 22. Preferably, the plastic film may have moisture permeability (air permeability). This improves water resistance of the back sheet 23 and also improves the wearer's comfortability while preventing stuffiness inside the absorbent product 1. Examples of the non-woven fabric overlaid on the plastic film include water-repellent non-woven fabrics or liquid-impermeable nonwoven fabrics. This improves the feel of the back sheet 23 such as touch and texture. The nonwoven fabric may, for example, be a spun-bonded nonwoven fabric or a melt-blown nonwoven fabric, which are formed of hydrophobic fiber, or may be an SMS (spun-bonded, melt-blown, and spun-bonded) nonwoven fabric and may be subjected to water-repellent processing as necessary.

The absorbent core 22 is an approximately sheet-like member whose thickness is smaller than its length and width. The absorbent core 22 is provided across the front, central, and rear parts of the absorbent product 1. That is, the absorbent core 22 is positioned on the belly side, the inseam, and the back side of the wearer. The shape of the absorbent core 22 in plan view may be modified in various ways. For example, the width of the rear part of the absorbent core 22 may be greater than the width of the front part. Alternatively, the width of the central part of the absorbent core 22 may be smaller than the widths of the front and rear parts.

The absorbent core 22 may be configured by, for example, dispersing a high-absorbent material into a sheet fiber assembly. The fiber assembly may, for example, be an assembly of pulp fiber and/or synthetic fiber. The high-absorbent material may, for example, be a particulate SAP (super absorbent polymer) or a fibrous SAF (super absorbent fiber). The absorbent core 22 may also be a sheet fiber assembly that does not contain any high-absorbent material. As another alternative, the absorbent core 22 may be a substance that substantially does not contain any fiber such as pulp fiber and that contains a high-absorbent material fixedly attached between two liquid-permeable sheet members (e.g., nonwoven fabrics) with a hot-melt adhesive or the like.

Each gather sheet 32 includes a side sheet 321 and elastic members 3221 to 3223 as shown in FIGS. 1 to 3. In FIGS. 1 and 3, reference signs 321 and 3221 to 3223 are given to only the gather sheet 32 on the right side. The side sheet 321 is an appropriately strip sheet member that is long in the longitudinal direction. In the present embodiment, the gather sheets 32 on the right and left sides are each provided with the three elastic members 3221 to 3223. Each of the elastic members 3221 to 3223 has a linear shape extending in the longitudinal direction and is fixedly attached to an inner region of the side sheet 321 in the width direction. While the absorbent product 1 is spread flat as shown in FIG. 1, the elastic members 3221 to 3223 are stretched, and when the elastic members 3221 to 3223 shrink, the gather sheets 32 shrink and the absorbent product 1 is curved in an approximately cup shape as shown in FIG. 2. As a result, the inner portions of the gather sheets 32 in the width direction stand up from the center sheet 31 as shown in FIG. 3.

FIG. 5 is a diagram showing the vicinity of the gather sheet 32 on the right side in FIG. 3 in enlarged dimensions. The gather sheet 32 on the left side also has the same structure. As shown in FIG. 5, the side sheet 321 has a structure in which an inside sheet 331 and an outside sheet 332 are overlaid one above the other by folding a single sheet. The folding position is at an inner edge 333 of the gather sheet 32 in the width direction. The three elastic members 3221 to 3223 are arranged between the inside sheet 331 and the outside sheet 332. Hereinafter, the three elastic members 3221 to 3223 are also referred to as the “first elastic member 3221,” the “second elastic member 3222,” and the “third elastic member 3223” in order from the inner side to the outer side in the width direction. The first elastic member 3221 is arranged along and in the vicinity of the edge 333 of the gather sheet 32. The second and third elastic members 3222 and 3223 are arranged and spaced outward in the width direction from the first elastic member 3221.

As the side sheet 321, a water-repellent or liquid-impermeable nonwoven fabric formed of hydrophobic fiber (e.g., a spun-bonded nonwoven fabric, a melt-blown nonwoven fabric, or an SMS nonwoven fabric) may be used and may be subjected to water-repellent processing as necessary. As the elastic members 3221 to 3223, for example, polyurethane yarn, a strip polyurethane film, or filiform or strip natural rubber may be used.

The side sheet 321 is fixedly attached on the center sheet 31. In FIGS. 1, 3, and 4, regions in which the center sheet 31 and the side sheets 321 are joined together, i.e., regions in which the center sheet 31 and the gather sheets 32 are joined together, are cross-hatched. In the present embodiment, the center sheet 31 and the pair of gather sheets 32 are joined together by welding involving pressure. Hereinafter, these joining regions are referred to as a “compression joining region 4.” The term “welding involving pressure” as used herein refers to, for example, thermal welding in which the sheets are pressed by the application of heat or to ultrasonic welding in which the sheets are pressed by the application of ultrasonic vibrations, and is so-called “embossing.” Since the compression is caused by pressure, “welding involving pressure” can also be regarded as “welding involving compression.” Note that the joints between the center sheet 31 and the gather sheet 32 may be made by using a different joining direction such as a method using a hot-melt adhesive in combination.

The compression joining region 4 includes central joining regions 41 that are positioned in the range of a central portion in the longitudinal direction and end joining regions 42 that are positioned in the ranges of both end portions in the longitudinal direction. As shown in FIG. 1, two central joining regions 41 and four end joining regions 42 exist within one absorbent product 1. Each central joining region 41 is spaced outward in the width direction from the three elastic members 3221 to 3223. Each end joining region 42 does not overlap with the three elastic members 3221 to 3223. As shown in FIG. 1, each central joining region 41 is long and slender in the longitudinal direction. Each end joining region 42 includes three linear regions that extend in the longitudinal direction. The three linear regions are hereinafter referred to as the “first linear region 421,” the “second linear region 422,” and the “third linear region 423” in order from the inner side to the outer side in the width direction. In FIG. 1, the reference signs 421 to 423 are given to only the linear regions shown in the upper right portion. The second linear region 422 and the third linear region 423 exist outward of the second and third elastic members 3222 and 3223 in the width direction. Thus, these linear regions are collectively referred to as an “outer joining region 432,” and the first linear region 421 that exists inward of the second and third elastic members 3222 and 3223 in the width direction is also referred to as an “inner joining region 431.”

The inner joining region 431 and the outer joining region 432 are spaced from each other in the width direction. The positions of the second and third elastic members 3222 and 3223 in the width direction are located between the inner joining region 431 and the outer joining region 432. The position of the innermost first elastic member 3221 in the width direction is located inward of the inner joining region 431 in the width direction. That is, the positions in the width direction of the three elastic members 3221 to 3223 overlap with none of the ranges of existence of the inner joining region 431 and the outer joining region 432 in the width direction.

In the manufacture of the absorbent product 1, a plurality of absorbent cores 22 are sandwiched between the top sheet 21 and the back sheet 23 that are both continuous and correspond to a plurality of absorbent products 1, and thereafter an individual absorbent product 1 is cut out and obtained from this layered continuum. Since the three elastic members 3221 to 3223 do not overlap with the end joining regions 42, the end portions of the three elastic members 3221 to 3223 after cutting freely shrink within the gather sheet 32. Therefore, the end joining regions 42 do not receive any force from the elastic members 3221 to 3223 after cutting. As a result, it is possible to prevent the end joining regions 42 hardened by the compression joint from being inwardly curved significantly, i.e., toward the wearer side, upon receipt of the force from the elastic members 3221 to 3223 and thereby to improve the wearing comfort of the absorbent product 1. Although, in FIG. 1, the ranges of existence of the end joining regions 42 in the longitudinal direction overlap with the ranges of existence of the elastic members 3221 to 3223 in the longitudinal direction, theses ranges of existence may not overlap with one another.

The second linear region 422 and the third linear region 423 are connected to each other on the inner side in the longitudinal direction. By dividing the outer joining region 432 into the second linear region 422 and the third linear region 423, it is possible to improve the flexibility of the absorbent product 1 in the regions positioned outward of the front and rear parts of the absorbent product 1 in the width direction. As a result, the regions positioned outward of the front and rear parts in the width direction can easily come along the inner surface of the diaper (including a diaper-shaped cover; the same applies below) or the body of the wearer, and accordingly wearing comfort is improved. Besides, the air permeability of the end joining regions 42 is also improved.

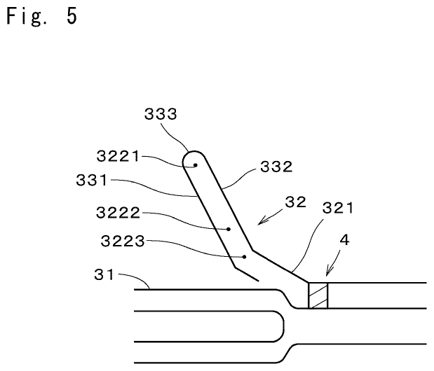

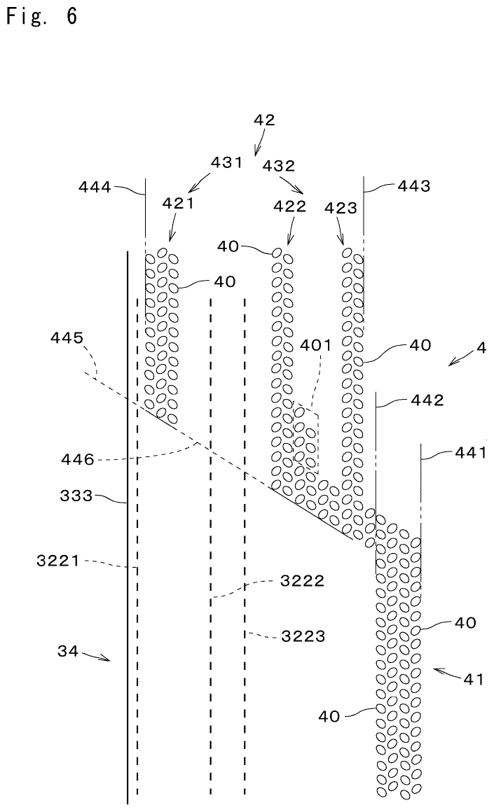

FIG. 6 is a diagram showing the vicinity of a boundary between one central joining region 41 and one end joining region 42 in the upper right portion of FIG. 1 in enlarged dimensions. In FIG. 6, a large number of dots 40 each represent one piece of the compression joining region 4, i.e., one dot-shaped embossment, and correspond to one recessed region. The dots 40 are hereinafter referred to as the “joining elements 40.” Although a large number of joining elements 40 are formed in the compression joining region 4, the intervals between the joining elements 40 are small and therefore a region that include spaces between the joining elements 40 is regarded as the compression joining region 4.

As indicated by a leader line in the upper portion in FIG. 6, an outer edge of the end joining region 42 in the width direction (at a position 443 in the width direction) is located in the width direction inward of the inner edge of the central joining region 41 in the width direction (at a position 442 in the width direction). In this way, by positioning the end joining region 42 inward of the central joining region 41 in the width direction, there are no portions hardened by the compression joints in the regions positioned outward of the front and rear parts of the absorbent product 1 in the width rection, and accordingly the flexibility is improved. As a result, the regions positioned outward of the front and rear parts in the width direction can easily come along the inner surface of the diaper or the body of the wearer, and accordingly, wearing comfort is improved. The central joining region 41 positioned outward of the end joining region 42 in the width direction allows the gathers 34 to stand up to a large degree and effectively prevents lateral leakage of the body wastes. Preferably, the dimension of the end joining region 42 in the width direction, i.e., the distance between the position 443 of the outer edge of the end joining region 42 and a position 444 of the inner edge of the end joining region 42, is greater than the dimension of the central joining region 41 in the width direction, i.e., the distance between a position 441 of the outer edge of the central joining region 41 and the position 442 of the inner edge thereof.

Here, the central joining region 41 and the outer joining region 432 are continuous in the longitudinal direction. This prevents the body wastes received by the absorbent product 1 from leaking out from the space between the central joining region 41 and the outer joining region 432. As described previously, the intervals of the joining elements 40 are narrow to such an extent that can prevent leakage of the body wastes. More preferably, the compression joining region 4 may exist along the overall longitudinal length of the top sheet 21. In this case, when the compression joining region 4 is formed using ultrasonic vibrations, the ultrasonic vibrations continuously act on the projections of a roll (so-called embossing roll) that form the compression joining region 4. As a result, the ultrasonic vibrations stably act on the projections of the embossing roll and reduces variations in the strength of the welding joints. Besides, the wearing away of the projections of the embossing roll is suppressed, and the life of the embossing roll is improved. When the compression joining region 4 is formed by thermal welding as well, it is possible to suppress variations in linear pressure generated between the pair of rolls that sandwich the sheet on which the compression joining region 4 is formed, and to reduce variations in the strength of the welding joints. Besides, the wearing away of the projections of the embossing roll is suppressed, and the life of the embossing roll is improved.

More preferably, a maximum value for the total length of the compression joints in the compression joining region 4 in the width direction is 1.25 times or more and 4 times or less of a minimum value therefor. Here, assuming a straight line extending in the width direction, the term “total length of the compression joints” as used herein refers to the total length of the ranges in which the compression joints are actually made on the straight line by the joining members 40 or the like (when there is only one range, the length of this range). More preferably, the total length of the compression joints may be 1.5 times or more and 3 times or less. This allows the ultrasonic vibrations to stably act on the projections of the embossing roll and thereby reduces variations in the strength of the welding joints. Besides, the wearing away of the projections of the embossing roll is suppressed, and the life of the embossing roll is improved. In the case where the compression joining region 4 is formed by thermal welding as well, it is possible to suppress variations in linear pressure generated between the pair of rolls that sandwich the sheet on which the compression joining region 4 is formed, and thereby to reduce variations in the strength of the welding joint. Besides, the wearing away of the projections of the embossing roll is suppressed, and the life of the embossing roll is improved.

The edge of the end joining region 42 on the central side in the longitudinal direction (the line denoted by a reference sign 445 in FIG. 6; portions corresponding to the edge of the end joining region 42 are indicated with solid lines, and portions other than the edge are indicated with broken lines) is a discontinuous line that is interrupted at the positions of the second and third elastic members 3222 and 3223. The edge is inclined toward the center in the longitudinal direction as the edge approaches outward in the width direction. Note that the edge is not limited to a straight line and may be a curve. An additional joining region 401 exists outward of the range in which the edge (denoted by a reference sign 446 in FIG. 6) is interrupted in the width direction. This reduces variations in the range of the projections of the embossing roll on which the ultrasonic vibrations act during rotation of the embossing roll, and accordingly reduces variations in the strength of the welding joints. Besides, the wearing away of the projections of the embossing roll is suppressed, and the life of the embossing roll is improved. In the case where the compression joining region 4 is formed by thermal welding as well, it is possible to suppress variations in linear pressure generated between the pair of rolls and thereby to reduce variations in the strength of the welding joints. Besides, the wearing away of the projections of the embossing roll is suppressed, and the life of the embossing roll is improved. Note that the “additional joining region” can be grasped as, for example, distortion of the edge that originally ought to exist linearly or as a joining region that exists independently, from the shape around the end joining region 42. The additional joining region 401 may be provided inward of the interrupted range 446 of the edge in the width direction.

Now, for the sake of convenience, the edge of the central joining region 41 that corresponds to the outer position 441 of the outermost joining element 40 in the width direction in the central joining region 41 is referred to as the “outer edge 441,” the edge of the central joining region 41 that corresponds to the inner position 442 of the innermost joining element 40 in the width direction is referred to as the “inner edge 442,” the edge of the end joining region 42 that corresponds to the outer position 443 of the outermost joining element 40 in the width direction in the end joining region 42 is referred to as the “outer edge 443,” and the edge of the end joining region 42 that corresponds to the inner position 444 of the innermost joining element 40 in the width direction is referred to as the “inner edge 444.” The ratio of existence of the joining elements 40 between the outer edge 443 and the inner edge 444 of the end joining region 42 is lower than the ratio of existence of the joining elements 40 between the outer edge 441 and the inner edge 442 of the central joining region 41. The term “ratio of existence of the joining elements 40” as used herein refers to the ratio of the number of joining elements 40 or the total length of the range of existence of the joining elements 40 to the width of the compression joining region 4 when the compression joining region 4 is cut in a straight line extending in the width direction. Note that the “ratio of existence of the joining elements 40” may be the ratio of the area of the joining elements 40 to the area of a target region as a whole.

Since the ratio of existence of the joining elements 40 in the end joining region 42 is lower than the ratio of existence of the joining elements 40 in the central joining region 41, the joining elements 40 are non-densely dispersed in the end joining region 42. This improves the flexibility of the absorbent product 1 in the regions of the front and rear parts of the absorbent product 1 and, in particular, the regions positioned outward of the front and rear parts in the width direction. As a result, the wearing comfort of the regions of the front and rear parts is improved. Meanwhile, in the central joining region 41, the gather sheet 32 can be firmly and fixedly attached to the center sheet 31. This prevents lateral leakage of the body wastes.

As shown in FIG. 6, the first elastic member 3221 located at the innermost position in the width direction is positioned inward of the inner joining region 431 (first linear region 421) in the width direction. This prevents an unnecessary force caused by the first elastic member 3221 from acting on the inside of the first linear region 421 in the width direction while forming ridges and grooves up to the inner edge 333 of the gather sheet 32.

A portion of the gather sheet 32 that is positioned inward of the compression joining region 4 in the width direction is not fixedly attached on the center sheet 31. This portion of the gather sheet 32 can be spaced above from the center sheet 31 (i.e., toward the wearer side when the absorbent product 1 is worn). As shown in FIGS. 1 and 3, the first elastic member 3221 is joined to the vicinity of the inner edge 333 of each side sheet 321 with an adhesive such as a hot-melt adhesive while being stretched and extending in the longitudinal direction. The second and third elastic members 3222 and 3223 are joined to the side sheet with an adhesive such as a hot-melt adhesive while being stretched and extending in the longitudinal direction and being spaced outward in the width direction from the first elastic member 3221. The second elastic member 3222 and the third elastic member 3223 are also spaced apart from each other in the width direction. When the absorbent product 1 is worn, the portion of each gather sheet 32 positioned inward of the central joining region 41 in the width direction is spaced above from the center sheet 31 and stands up toward the wearer side due to the shrinkage of the elastic members 3221 to 3223 as shown in FIG. 3. The shrinkage of the elastic members 3221 to 3223 produces the gathers 34 in the portion of the gather sheet 32 that is positioned inward of the central joining region 41 in the width direction. This allows the absorbent product 1 to prevent leakage of urine or the like from around the legs. Note that the side sheet 321 and the elastic members 3221 to 3223 may be joined together by using a technique other than an adhesive, and for example, they may be joined together by welding using heat or ultrasonic vibrations.

The absorbent product 1 may be modified in various ways. For example, the back sheet 23 is not limited to a single sheet and may be configured by overlaying a plurality of sheets one above another, or may be a sheet obtained by joining a plurality of sheets together in the longitudinal or width direction. The back sheet 23 may have a function of preventing leakage of a liquid in the absorbent core 22 to the outside of the absorbent product 1. Typically, the back sheet 23 may include a plastic film provided with an infinite number of fine holes, and a nonwoven fabric joined to the outer surface of the plastic film. The expression for the “back sheet 23” means a sheet that covers the lower surface of the absorbent core 22, i.e., the surface on the side opposite to the wearer.

The expression for the “top sheet 21” means a sheet that covers the upper surface of the absorbent core 22, i.e., the surface facing the wearer. While the top sheet 21 of the disposable absorbent product 1 according to the present embodiment includes the center sheet 31 and the gather sheets 32, the top sheet 21 may further include different sheets other than those described above. The center sheet 31 has liquid permeability. However, there is no need for the center sheet 31 as a whole to have liquid permeability. The center sheet 31 is also not limited to a single sheet, and may be configured by overlaying a plurality of sheets one above another, or may be a sheet obtained by joining a plurality of sheets together in the longitudinal or width direction.

The gather sheets 32 mean the sheets including the gathers 34 that stand up toward the groin of the wearer, and the structure and shape of the gather sheets 32 are not limited to the examples described above. The gather sheets 32 may be joined to the lower surface of the center sheet 31 by welding involving pressure. In this case, for example, the gather sheets 32 may be joined to the lower surface of the center sheet 31 in the central joining region 41, and the portions of the gather sheets 32 that are folded upward at the edges of the center sheet 31 may be joined to the upper surface of the center sheet 31 in the end joining regions 42.

The side sheet 321 of each gather sheet 32 is not limited to the sheet configured by folding a single sheet and may be configured by overlaying a plurality of sheets one above another, or may be a sheet obtained by joining a plurality of sheets together in the longitudinal or width direction. The gather sheets 32 prevent leakage of the body wastes to the outside in the width direction, and at least the insides of the gather sheets 32 have hydrophobicity. Although the gather sheets 32 in FIG. 3 stand up from the outside toward the inside in the width direction, they may be folded outward in the width direction in the central joining region 41 and then stand up toward the groin of the wearer.

The number of elastic members included in each gather sheet 32 may be one, may be two, or may be four or more. That is, each gather sheet 32 is provided with at least one elastic member. Preferably, the elastic member(s) may be linear. The expression “linear” includes meaning of “thin filiform,” “flat-sectioned filiform,” and “flat-sectioned tape.” In any case, it is preferable that at least one elastic member does not overlap with the end joining regions 42. To be more precise, the position of at least one elastic member in the width direction does not overlap with the range of existence of the end joining regions 42 in the width direction. This reduces the possibility that the shrinkage force of at least one elastic member may act on the end joining regions 42. Preferably, one elastic member may be arranged along and in the vicinity of the inner edge 333 of the side sheet 321 in the width direction. The elastic members may be fixedly attached to the side sheet 321 by welding using heat or ultrasonic vibrations.

In the case where the number of elastic members is one, the outer joining region 432 is a region that exists outward of the elastic member in the width direction. The inner joining region 431 is a region that exists inward of the elastic member in the width direction. In the case where the number of elastic members is two or more, the outer joining region 432 is a region that exists outward of any of the elastic members in the width direction. The inner joining region 431 is a region that exists inward of the any of the elastic members in the width direction. To be expressed more generally, the outer joining region 432 is a region that exists outward of any elastic member among at least one elastic member (where the number of the any elastic member may be one or more) in the width direction, and the inner joining region 431 is a region that exists inward of the any elastic member in the width direction. Since the end joining regions 42 do not overlap with any of the elastic members, the end joining regions 42 will not receive any force directly from the elastic members. This prevents the end joining regions 42 hardened by the compression joints from being inwardly curved significantly (i.e., toward the viewer of the drawing in FIG. 1), i.e., toward the wearer, and accordingly improves the wearing comfort of the absorbent product 1. In other words, it is possible to reduce wearing discomfort when the absorbent product 1 is worn, as compared to the case where the end joining regions 42 overlap with the elastic members.

The presence of the inner joining region 431 and the outer joining region 432 and the presence of one or more (preferably, two as in the above-described embodiment) elastic members arranged in the width direction between the inner joining region 431 and the outer joining region 432 allows the portion of each gather sheet 32 in which the gathers 34 are formed to stand up easily. The presence of at least one elastic member between the inner joining region 431 and the outer joining region 432 stabilizes the overall shape of the gathers 34. In the gather sheet 32, in order to allow the ends of the gathers 34 to stand up with stability, it is preferable that the elastic member positioned at the innermost position in the width direction among a plurality of elastic members is positioned inward of the inner joining region 431 in the width direction. Moreover, in order to position the inner joining region 431 as far inward as possible, it is preferable that the elastic member positioned at the second innermost position from the inside in the width direction among the plurality of elastic members may be positioned between the inner joining region 431 and the outer joining region 432 in the width direction. That is, it is preferable that the number of elastic members positioned inward of the inner joining region 431 in the width direction may be one.

At least one elastic member may be provided in various ways on the gather sheet 32, and in any case, the range(s) of existence of the elastic member(s) in the longitudinal direction may or may not overlap with the range of existence of the end joining region 42 in the longitudinal direction.

In the above-described embodiment, the compression joining region 4 corresponds to a large number of dot-shaped joining elements 40, but it may also be a large number of linear joining elements or may have a mesh joining shape. Of course, the center sheet 31 and each gather sheet 32 may be welded together without any clearance across the compression joining region 4. In any case, it is preferable that a maximum value for the total length of the compression joints in the width direction in the compression joining region 4 may be 1.25 times or more and 4 times or less of the minimum value therefor. More preferably, the above maximum value for the total length may be 1.5 times or more and 3 times or less of the minimum value therefor.

Preferably, the compression joining region 4 may exist continuously from the front joining region 42 to the rear joining region 42. More preferably, the compression joining region 4 may exist along the overall length of the top sheet 21 in the longitudinal direction. The expression that the compression joining region “exists continuously” as used herein includes the case where the joining elements 40 are spaced apart from one another to such a degree that does not cause leakage of the body wastes. Moreover, the compression joining region 4 does not always have to exist continuously, and the center sheet 31 and the gather sheet 32 may be joined together with an adhesive at positions where the compression joining region 4 does not exist. In the above-described embodiment, each end joining region 42 is divided into the inner joining region 431 and the outer joining region 432, but the inner joining region 431 may be omitted. In this case, the outer joining region 432 corresponds with the end joining region 42, and it is preferable that the outer joining region 432 and the central joining region 41 may be continuous in the longitudinal direction, and accordingly, the compression joining region 4 as a whole is continuous in the longitudinal direction.

In the above-described embodiment, the outer edge 443 of each end joining region 42 in the width direction is positioned, in the width direction, inward of the inner edge 442 of the central joining region 41 in the width direction, but the outer edge 443 of the end joining region 42 in the width direction may be positioned, in the width direction, inward of only the outer edge 441 of the central joining region 41 in the width direction. In this case as well, it is possible to improve the flexibility of the outer portion(s) of the front and/or rear parts of the absorbent product 1 in the width direction. Since there is a case where the compression joining region 4 is an assembly of dot-shaped joining elements 40, the edges 441 to 444 mean, as described above, lines that are in contact with the inner and outer sides of the compression joining region 4. In the case where the edges 441 to 444 are curves, the language saying that one edge is positioned inward of another edge in the width direction means in principle that the one edge as a whole is positioned inward of the other edge as a whole in the width direction, but it also includes the case where an extremely small portion of the one edge or an extremely small portion of the other edge does not satisfy the above definition.

In the above-described embodiment, the outer joining region 432 includes the second linear region 422 and the third linear region 423 that extend in the longitudinal direction, but the outer joining region 432 may be configured by three or more linear regions. These linear regions do not necessarily have to be connected to one another. In this case, the outermost linear region may be regarded as the outer joining region 432. Forming the end joining region 42 as an assembly of linear regions improves the flexibility of the end joining region 42 and improves air permeability in the end joining region 42. It is preferable that at least a portion of the end joining region 42 on the end side in the longitudinal direction may be an assembly of a plurality of linear regions extending in the longitudinal direction.

Preferably, the center sheet 31 and the pair of gather sheets 32 may be joined together only in the compression joining region 4. That is, they may be joined together by only welding, and there is no adhesive applied therebetween. Of course, an adhesive may be used in an auxiliary manner as necessary when the center sheet 31 and the pair of gather sheets 32 are joined together.

The above-described absorbent product 1 is merely an example, and the joint made by welding between the center sheet 31 and the gather sheets 32 described in the above embodiment may be utilized as a pants-type disposable diaper, an open-type disposable diaper that is set to around the waist with tapes, an auxiliary absorbent pad arranged in a diaper or a diaper-shaped cover, a urine pad, or any of other various absorbent products that receive body wastes from a wearer.

The configurations of the above-described preferred embodiment and variations may be appropriately combined as long as there are no mutual inconsistencies.

While the invention has been shown and described in detail, the foregoing description is in all aspects illustrative and not restrictive. It is therefore understood that numerous modifications and variations can be devised without departing from the scope of the invention.

### Reference Signs List

1 absorbent product

4 compression joining region

11 center line

21 top sheet

22 absorbent core

23 back sheet

31 center sheet

32 gather sheet

40 joining element

41 central joining region

42 end joining region

321 side sheet

401 additional joining region

421 first linear region

422 second linear region

423 third linear region

431 inner joining region

432 outer joining region

441 (position of) outer edge (of central joining region)

442 (position of) inner edge (of central joining region)

443 (position of) outer edge (of end joining region)

444 (position of) inner edge (of end joining region)

445 line (along edge on center side of end joining region)

3221 first elastic member

3222 second elastic member

3223 third elastic member

1. A disposable absorbent product for receiving a body wastes from a wearer, comprising:
a top sheet;
a back sheet; and
an absorbent core positioned between said top sheet and said back sheet,
wherein said top sheet includes:
a center sheet; and
a pair of gather sheets fixedly attached to said center sheet while being spaced apart from a center line extending in a longitudinal direction on both sides in a width direction perpendicular to said longitudinal direction,
each of said pair of gather sheets includes:
a side sheet that is long in said longitudinal direction; and
an elastic member extending in said longitudinal direction and fixedly attached to a region on an inner side in said width direction of said side sheet,
said center sheet and said pair of gather sheets are joined together in a compression joining region by welding involving pressure,
said compression joining region between said center sheet and each gather sheet includes:
a central joining region positioned in a central range in said longitudinal direction and spaced outward in said width direction from said elastic member; and
an end joining region that is positioned in ranges of both end portions in said longitudinal direction and does not overlap with said elastic member, and
said end joining region includes:
an outer joining region located outward of said elastic member in said width direction; and
an inner joining region located inward of said elastic member in said width direction.

2. The disposable absorbent product according to claim 1, wherein
said central joining region and said outer joining region are continuous in said longitudinal direction.

3. The disposable absorbent product according to claim 1, wherein
an outer edge of said end joining region in said width direction is located inward in said width direction of an inner edge in said width direction of said central joining region.

4. The disposable absorbent product according to claim 1, wherein
said compression joining region is an assembly of a large number of dot-shaped joining elements, and
a ratio of existence of joining elements between outer and inner edges in said width direction of said end joining region is lower than a ratio of existence of joining elements between outer and inner edges in said width direction of said central joining region.

5. The disposable absorbent product according to claim 1, comprising:
a plurality of elastic members including said elastic member,
wherein an elastic member positioned on an innermost side in said width direction among said plurality of elastic members is positioned inward in said width direction of said inner joining region.

6. The disposable absorbent product according to claim 5, wherein
an elastic member positioned on a second innermost side in said width direction among said plurality of elastic members is positioned between said inner joining region and said outer joining region in said width direction.

7. The disposable absorbent product according to claim 1, wherein
said outer joining region includes a plurality of linear regions extending in said longitudinal direction.

8. The disposable absorbent product according to claim 1, wherein
said compression joining region exists along an overall length in said longitudinal direction of said top sheet, and
a maximum value of a total length of a compression joint in said width direction of said compression joining region is 1.25 time or more and 4 times or less of a minimum value of the total length.

9. The disposable absorbent product according to claim 1, wherein
an edge of said end joining region on a central side in said longitudinal direction is a discontinuous line,
said edge is inclined toward a center in said longitudinal direction toward an outside in said width direction, and
an additional joining region exists on an outer or inner side in said width direction where said edge is interrupted.

10. The disposable absorbent product according to claim 1, being arranged in a diaper or in a diaper-shaped cover.
